(19) United States
(12) Patent Application Publication
(10) Pub. No.: US 2024/0417812 A1
(43) Pub. Date: Dec. 19, 2024

(54) METHODS FOR TREATING SOLID CANCER PATIENTS WITH CLONAL HEMATOPOIESIS OF INDETERMINATE POTENTIAL

(71) Applicant: Olatec Therapeutics, Inc., New York, NY (US)

(72) Inventors: Charles A. Dinarello, Boulder, CO (US); Isak Tengesdal, Menlo Park, CA (US)

(21) Appl. No.: 18/820,156

(22) Filed: Aug. 29, 2024

Related U.S. Application Data
(63) Continuation of application No. PCT/US2023/063452, filed on Mar. 1, 2023.
(60) Provisional application No. 63/268,793, filed on Mar. 2, 2022.

(51) Int. Cl. C12Q 1/6886; A61K 31/275; A61K 39/00; A61P 35/00; C07K 16/28

(52) U.S. Cl. CPC C12Q 1/6886; A61K 31/275; A61P 35/00; C07K 16/2818; A61K 2039/505; C12Q 2600/106; C12Q 2600/156

(57) ABSTRACT

The present invention is directed to a method for treating solid cancer in patients with clonal hematopoiesis of indeterminate potential (CHIP). The method introduces a therapeutic intervention of dapansutrile to improve the treatment of a solid cancer, such as breast cancer, in a CHIP patient. The method includes first determining whether a patient has a CHIP condition by detecting the presence or absence of Tet2 or DNMT3A mutation from a biological sample of the patient, and then followed by a therapeutic intervention with dapansutrile in patients having a CHIP condition. This application shows a role for CHIP in driving breast cancer. The data provide a rationale for Tet2 or DNMT3A mutation screening in breast cancer patients and offer dapansutrile as a therapeutic treatment.

### CROSS-REFERENCE TO RELATED APPLICATIONS

This application is a continuation of PCT/US2023/063452, filed Mar. 1, 2023; which claims priority to U.S. Provisional Application No. 63/268,793, filed Mar. 2, 2022. The contents of the above-identified applications are incorporated herein by reference in their entireties.

### TECHNICAL FIELD

The present invention relates to methods for treating solid cancer in patients with clonal hematopoiesis of indeterminate potential (CHIP). The method includes first determining whether a patient has a CHIP condition by detecting the presence or absence of Tet2 or DNMT3A mutation, and then followed by a therapeutic intervention with dapansutrile in the patient having a CHIP condition.

### BACKGROUND

As people age, their tissues accumulate an increasing number of somatic mutations. When this process happens in the hematopoietic system, a substantial proportion of circulating blood cells may derive from a single mutated stem cell. This outgrowth, called “clonal hematopoiesis,” is highly prevalent in the elderly population. Clonal hematopoiesis (CH) refers to the gradual, selective expansion of hematopoietic stem cells (HSCs) harboring somatic mutations, often in one allele of a gene. CH is characterized by the over-representation of blood cells derived from a single clone. Blood cancers such as chronic myeloid leukemia or myelodysplastic syndromes are prototypical examples of clonal hematopoiesis. However, the same mutations found in these cancers are also seen in a large proportion of the healthy elderly population.

Clonal hematopoiesis of indeterminate potential (CHIP) refers to the presence of clonal molecular genetic or cytogenetic changes in blood or bone marrow cells in the absence of signs of hematological neoplasm and absence of cytopenia. CHIP is a non-malignant condition characterized by mutation and clonal expansion of blood cells. The incidence of CHIP increases with age. CHIP is diagnosed when a test on a person's blood or bone marrow sample shows that blood cells are carrying one of the genetic mutations associated with the condition.

CHIP is associated with a higher risk of developing hematological malignancies and cardiovascular disease, as well as a reduced lifespan.

Tumorigenesis is initiated by genomic alterations including point mutations, gene deletion, chromosomal rearrangements leading to cell transformation, self-sufficient proliferation, insensitivity to anti-proliferative signals, evasion of apoptosis and unlimited replicative potential, leading ultimately to tissue invasion and metastasis. However, expansion of tumor cells is linked to a complex network of events that involve both cancer and non-cancer cells. Chronic inflammation is a classic example of such promoting conditions (1, 2).

The pro-inflammatory cytokine IL-1β is a potent mediator of many chronic inflammatory diseases (3). Consistent with the linkage of cancer to chronic inflammation, it has been shown that IL-1β is over-expressed in several tumors and functions as an inducer of tumor promoting mechanisms including angiogenesis, immunosuppression, recruitment of tumor-associated macrophages (TAMs) and metastasis (4-6).

Types of breast cancer include ductal carcinoma in situ (DCIS), invasive ductal carcinoma (IDC), triple negative breast cancer (TNBC), inflammatory breast cancer (IBC), metastatic breast cancer, and breast cancer during pregnancy, among other types. Triple negative breast cancer tumors are characterized by an absence of estrogen receptors (ER), progesterone receptors (PR), and elevated human epidermal growth factor receptor 2 (HER2) protein levels (7).

NLRP3 (NOD-like receptor family, pyrin domain containing 3), also known as NLRP3 or cryopyrin, is one of the sensors of the inflammasome, a macromolecular structure involved in interleukin-1β (IL-1β) and IL-18 processing. NLRP3 senses intracellular danger during intracellular infections (bacterial and viral proteins) or tissue injury (ischemia). NLRP3 activation leads to recruitment of ASC (apoptosis-associated speck-like protein containing carboxyterminal caspase recruitment domain) and caspase-1 leading to inflammasome formation and ultimately cell death.

Dapansutrile is a small, synthetic molecule of β-sulfonyl nitrile which has been demonstrated to selectively inhibit the NLRP3 inflammasome and be safe when orally administered to healthy subjects (8).

### BRIEF DESCRIPTION OF THE DRAWINGS

FIG. 1A shows tumor growth curves from germline Tet2+/+ (wild type) or Tet2^{+/-} mice orthotopically implanted with E0771 cells. Tet2^{+/-} mice was orally treated with dapansutrile (OLT1177®) or control food without dapansutrile (N=8, two independent experiments).

Tet2+/− driven breast cancer progression is NLRP3 activation-dependent. FIG. 1B shows Mean±SEM of IL-1β production from bone marrow adherent cells isolated from tumor-bearing mice.

FIG. 2. Bone marrow (BM) chimeras were generated by transplanting 75% WT: 25% Tet2+/+ or Tet2+/− mixed BM. BM chimera mice were orthotopically implanted with E0771 breast tumors and given standard or dapansutrile diet. FIG. 2 shows tumor growth curve from Tet2+/+ (WT) or Tet2+/− chimeric mice implanted with E0771 cells receiving standard or dapansutrile diet (N=10, two independent experiments).

FIGS. 3A-3B show total percent of donor-derived cells in peripheral blood (3A) and tumor (3B) of BM chimera mice from FIG. 2.

FIGS. 4A-4B show frequency (4A) and gMFI of MHC-II+ expression (4B) on tumor-infiltrating granulocytes of BM chimera mice from FIG. 2.

FIGS. 5A-5B show plasma cytokine production of chemokine C-C motif ligand 2 (CCL2, 5A) and keratinocyte chemoattractant (KC, 5B) of BM chimera mice from FIG. 2

In all figures: *** p<0.001, ** p<0.01, *p<0.05.

### DETAILED DESCRIPTION

Clonal hematopoiesis (CH) is associated with increased hematologic malignancies. CH can be defined as somatic mutations in hematopoietic stem cells resulting in clonal expansion of myeloid cells with an increased inflammatory phenotype. Driving mutations in Ten-eleven translocation 2 (Tet2) and/or DNA methyltransferase 3a (DNMT3A), which are genes involved in DNA methylation, lead to clonal expansion. Tet2 is one of the three enzymes responsible for catalyzing the oxidation of 5-methylcytosine to 5-hydroxymethylcytosine and further intermediates, which can eventually lead to demethylation. The DNMT3A gene provides instructions for making DNA methyltransferase 3a, which is one of the two enzymes responsible for de novo methylation of the fifth position in cytosine bases of DNA, a mark that influences gene expression.

The inventors have discovered a role for CHIP in driving a solid cancer such as breast cancer and prostate cancer. In solid tumor infiltrating myeloid cells, immunosuppressive tumor-microenvironments (TME) are promoted through dysregulated cytokine signaling. The link between CHIP and solid tumors is that the infiltrating myeloid cells show up to the TME and have a hyperinflammatory response, furthering the tumor promoting signaling. For a cancer patient having CHIP, the disease is likely to be more severe or the patient is at greater risk. In breast cancers, CHIP has detrimental effects in all advanced stage breast cancers, this is because when breast cancer progresses, IL-1β increases with disease severity.

The proinflammatory cytokine IL-1β promotes breast cancer progression and correlates with disease severity. Activation of the NLRP3 inflammasome in myeloid cells is the main source of IL-1β processing.

Activation of the NLRP3 inflammasome amplifies the inflammatory response to tissue injury and mediates further damage. Dapansutrile is a selective NLRP3 inflammasome inhibitor; dapansutrile reduces inflammation by preventing activation of the NLRP3 inflammasome.

In view of the mechanism of action of dapansutrile, which prevents production and/or release of IL-1β and IL-18 and inhibits the formation of NLRP3 inflammasome in animals and human subjects, the inventors have discovered a method for introducing a therapeutic intervention of dapansutrile to improve the treatment of a solid cancer, such as breast cancer or prostate cancer in a CHIP patient. The method includes first determining whether a patient has a CHIP condition by detecting the presence or absence of Tet2 or DNMT3A mutation, and then followed by a therapeutic intervention with dapansutrile in patients having a CHIP condition.

### Method of Treatment

The present invention is directed to a method of treating a solid cancer such as breast cancer or prostate cancer a patient. The method comprises the steps of: (a) determining clonal mutation of TET2 or DNMT3A in a sample of a patient that has a solid cancer and is treated with a non-dapansutrile treatment, and (b1) treating the patient with an effective amount of dapansutrile in addition to the non-dapansutrile treatment, if the patient has a variant allele frequency (VAF) of TET2 or DNMT3A greater than 0.02, or (b2) continuing treating the patient with the non-dapansutrile treatment, without dapansutrile treatment, if the patient has a VAF of TET2 or DNMT3A≤0.02.

In the present method, a patient that has a solid cancer and is treated with a non-dapansutrile treatment is identified, and a biological sample from the patient is obtained. The biological sample, for example, can be a blood sample or bone marrow cells from the patient. A blood sample is preferred. Clonal mutation of TET2 or DNMT3A in patient's sample is then determined.

TET2 and DNMT3A are the most commonly mutated CHIP-driver genes. Monocytes and T cells with TET2 or DNMT3A mutation show very similar increase of proinflammatory gene expression.

Determining clonal mutation of TET2 or DNMT3A in the sample of a cancer patient provides prognosis of the patient. The inventors have discovered that patients with high Tet2 or DNMT3A VAF are at greater risk of advancing to late-stage disease and are benefited by dapansutrile treatment. Dapansutrile intervention therapy reverses CHIP phenotype and may provide some synergistic effect with other non-dapansutrile treatment by inhibition of NLRP3 activation that attenuates the efficacy of other treatment.

For determining clonal mutation, the threshold for CHIP is set at a variant allele fraction (VAF) of Tet2 or DNMT3A of 2% (meaning 2% of the sequenced alleles contain the mutation, or roughly 4% of the cells contain the mutation, assuming the mutation is heterozygous). VAF of Tet2 or DNMT3A can be detected by next generation sequencing or error-corrected sequencing from patient's blood sample (9, 10). For example, VAF can be detected by sample preparation, DNA isolation, target enrichment, high-throughput sequencing, and variant calling as described in Dorsheimer (10).

After the clonal mutation of TET2 or DNMT3A is determined, if the patient has a variant allele frequency (VAF) of TET2 or DNMT3A greater than 0.02, the patient is treated with an effective amount of dapansutrile in addition to the non-dapansutrile treatment. If the patient has a VAF of TET2 or DNMT3A≤0.02, the patient is continued with the non-dapansutrile treatment, without adding the dapansutrile treatment.

“An effective amount of dapansutrile” as used herein, is the amount of dapansutrile effective to treat a disease by ameliorating the pathological condition, and/or reducing, improving, and/or eliminating the symptoms of the disease. For example, an effective amount is an amount that reduces the growth of cancer, and/or reduces the tumor size.

Solid cancer suitable to be treated by the present method, for example, includes breast cancer and prostate cancer. Breast cancer includes triple negative breast cancer (TNBC), ductal carcinoma in situ (DCIS), invasive ductal carcinoma (IDC), inflammatory breast cancer (IBC), metastatic breast cancer, and breast cancer during pregnancy, among other types.

Non-dapansutrile treatments of breast cancer that are suitable for dapansutrile intervention include, but not limited to, checkpoint inhibitor therapy, which is a form of cancer immunotherapy. Checkpoint inhibitor therapy targets immune checkpoints, key regulators of the immune system that when stimulated can dampen the immune response to an immunologic stimulus. Some cancers can protect themselves from attack by stimulating immune checkpoint targets. Checkpoint inhibitors currently used for treating breast cancer and suitable for dapansutrile intervention include, but not limited to, inhibitors to programmed cell death protein 1 (PD-1), programmed death ligand 1 (PD-L1), and cytotoxic T lymphocyte associated protein 4 (CTLA-4). PD-1 is found on the surface of T cells and is the receptor for PD-L1. PD-1 plays a role in down-regulating immune responses by suppressing inflammatory T cell activity. This mechanism helps the body to prevent autoimmune diseases, however, the mechanism can also prevent the cancer cells from being killed (11).

Non-dapansutrile treatments of breast cancer that are suitable for dapansutrile intervention may also include chemotherapy such as 5-fluorouridine and gemcitibine.

Non-dapansutrile treatments of prostate cancer that are suitable for dapansutrile intervention include, but not limited to, radiotherapy, chemotherapy, and/or long-term androgen deprivation therapy. Chemotherapeutic agents currently approved for prostate cancer are docetaxel or cabaxitaxel, these along with androgen deprivation have proven effective at increasing survival. Hormonal strategies targeting androgen production or signaling include abiraterone or enzalutamide.

Dapansutrile intervention therapy reverses CHIP phenotype and may provide some synergistic effect with chemotherapy by inhibition of NLRP3 activation that attenuates the efficacy of other treatment. For example, dapansutrile intervention therapy reverses CHIP phenotype and may provide some synergistic effect with the check point inhibitor treatment such as anti-PD-1 and anti-PD-L1.

If the patient is determined to have a variant allele frequency (VAF) of TET2 or DNMT3A greater than 0.02 and is suitable for the combination treatment of dapansutrile, the dapansutrile treatment and the non-dapansutrile treatment can be administered simultaneously or sequentially.

### Compound

The present invention uses purified dapansutrile (3-methanesulfonyl-propionitrile), or the pharmaceutically acceptable solvate thereof, as a therapeutic intervention.

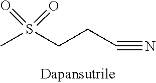

“Pharmaceutically acceptable solvates,” as used herein, are solvates that retain the desired biological activity of the parent compound and do not impart undesired toxicological effects. “Solvates,” as used herein, are addition complexes in which the compound is combined with an acceptable co-solvent in some fixed proportion. Co-solvents include, but are not limited to, water, acetic acid, ethanol, and other appropriate organic solvents.

### Pharmaceutical Composition

The active compound dapansutrile, or its pharmaceutically acceptable solvate in the pharmaceutical compositions in general is in an amount of about 0.1-5% for an injectable formulation, about 1-90% for a tablet formulation, 1-100% for a capsule formulation, about 0.01-20%, 0.05-20%, 0.1-20%, 0.2-15%, 0.5-10%, or 1-5% (w/w) for a topical formulation, and about 0.1-5% for a patch formulation.

“About” as used in this application, refers to +10% of the recited value.

Pharmaceutically acceptable carriers, which are inactive ingredients, can be selected by those skilled in the art using conventional criteria. Pharmaceutically acceptable carriers include, but are not limited to, non-aqueous based solutions, suspensions, emulsions, microemulsions, micellar solutions, gels, and ointments. The pharmaceutically acceptable carriers may also contain ingredients that include, but are not limited to, saline and aqueous electrolyte solutions; ionic and nonionic osmotic agents such as sodium chloride, potassium chloride, glycerol, and dextrose; pH adjusters and buffers such as salts of hydroxide, phosphate, citrate, acetate, borate; and trolamine; antioxidants such as salts, acids and/or bases of bisulfite, sulfite, metabisulfite, thiosulfite, ascorbic acid, acetyl cysteine, cystein, glutathione, butylated hydroxyanisole, butylated hydroxytoluene, tocopherols, and ascorbyl palmitate; surfactants such as lecithin, phospholipids, including but not limited to phosphatidylcholine, phosphatidylethanolamine and phosphatidyl inositiol; poloxamers and ploxamines, polysorbates such as polysorbate 80, polysorbate 60, and polysorbate 20, polyethers such as polyethylene glycols and polypropylene glycols; polyvinyls such as polyvinyl alcohol and povidone; cellulose derivatives such as methylcellulose, hydroxypropyl cellulose, hydroxyethyl cellulose, carboxymethyl cellulose and hydroxypropyl methylcellulose and their salts; petroleum derivatives such as mineral oil and white petrolatum; fats such as lanolin, peanut oil, palm oil, soybean oil; mono-, di-, and triglycerides; polymers of acrylic acid such as carboxypolymethylene gel, and hydrophobically modified cross-linked acrylate copolymer; polysaccharides such as dextrans and glycosaminoglycans such as sodium hyaluronate. Such pharmaceutically acceptable carriers may be preserved against bacterial contamination using well-known preservatives, these include, but are not limited to, benzalkonium chloride, ethylene diamine tetra-acetic acid and its salts, benzethonium chloride, chlorhexidine, chlorobutanol, methylparaben, thimerosal, and phenylethyl alcohol, or may be formulated as a non-preserved formulation for either single or multiple use.

For example, a tablet formulation or a capsule formulation of dapansutrile may contain other excipients that have no bioactivity and no reaction with the active compound. Excipients of a tablet may include fillers, binders, lubricants and glidants, disintegrators, wetting agents, and release rate modifiers. Binders promote the adhesion of particles of the formulation and are important for a tablet formulation. Examples of binders include, but not limited to, carboxymethylcellulose, cellulose, ethylcellulose, hydroxypropylmethylcellulose, methylcellulose, karaya gum, starch, starch, and tragacanth gum, poly(acrylic acid), and polyvinylpyrrolidone.

For example, a patch formulation of dapansutrile may comprise some inactive ingredients such as 1,3-butylene glycol, dihydroxyaluminum aminoacetate, disodium edetate, D-sorbitol, gelatin, kaolin, methylparaben, polysorbate 80, povidone, propylene glycol, propylparaben, sodium carboxymethylcellulose, sodium polyacrylate, tartaric acid, titanium dioxide, and purified water. A patch formulation may also contain skin permeability enhancer such as lactate esters (e.g., lauryl lactate) or diethylene glycol monoethylether.

Topical formulations including dapansutrile can be in a form of gel, cream, lotion, liquid, emulsion, ointment, spray, solution, and suspension. The inactive ingredients in the topical formulations for example include, but not limited to, lauryl lactate (emollient/permeation enhancer), diethylene glycol monoethylether (emollient/permeation enhancer), DMSO (solubility enhancer), silicone elastomer (rheology/texture modifier), caprylic/capric triglyceride, (emollient), octisalate, (emollient/UV filter), silicone fluid (emollient/diluent), squalene (emollient), sunflower oil (emollient), and silicone dioxide (thickening agent). In one embodiment, diethylene glycol monoethylether is included in the topical gel formulation.

The pharmaceutical composition of dapansutrile can be applied by systemic administration or local administration. Systemic administration includes, but is not limited to oral, parenteral (such as intravenous, intramuscular, subcutaneous or rectal), and inhaled administration. In systemic administration, the active compound first reaches plasma and then distributes into target tissues. Oral administration is a preferred route of administration for the present invention. Local administration includes topical administration.

Dosing of the composition can vary based on the extent of the subject's breast cancer and each patient's individual response. For systemic administration, plasma concentrations of the active compound delivered can vary; but are generally 1×10⁻¹⁰-1×10⁻⁴ moles/liter, and preferably 1×10⁻⁸-1×10⁻⁵ moles/liter.

In one embodiment, dapansutrile is administrated orally to a subject. The dosage for oral administration is generally at least 1 mg/kg/day and less than 100 mg/kg/day, preferably 5-100 mg/kg/day, depending on the subject's age and condition. For example, the dosage for oral administration is 1-10, or 1-50, or 1-100, or 5-50, or 5-100, or 10-50, or 10-100 mg/kg/day for a human subject. For example, the dosage for oral administration is 100-10,000 mg/day, and preferably 100-2500, 500-2500, 500-4000, 1000-5000, 2000-5000, 2000-6000, or 2000-8000 mg/day for a human subject. The drug can be orally taken once, twice, three times, or four times a day. The patient is treated daily for 14 days up to 1 month, 2 months, or 3 months or for lifespan.

In one embodiment, dapansutrile is administrated intravenously to a subject. The dosage for intravenous bolus injection or intravenous infusion is generally 0.03 to 5 or 0.03 to 1 mg/kg/day.

In one embodiment, dapansutrile is administrated subcutaneously to the subject. The dosage for subcutaneous administration is generally 0.3-20, 0.3-3, or 0.1-1 mg/kg/day.

In one embodiment, dapansutrile is applied topically. The topical dapansutrile composition is topically applied at least 1 or 2 times a day, or 3 to 4 times per day, depending on the medical issue and the disease pathology. In general, the topical composition comprises about 0.01-20%, or 0.05-20%, or 0.1-20%, or 0.2-15%, 0.5-10, or 1-5% (w/w) of the active compound. Typically, 0.2-10 mL of the topical composition is applied to the individual per dose.

Those of skill in the art will recognize that a wide variety of delivery mechanisms of dapansutrile may be suitable for the present invention.

When a breast cancer patient or a prostate cancer patient is determined to be additionally treated with dapansutrile, the non-dapansutrile treatment of the patient will follow the same protocol already established for the patient. Alternatively, the dosage or treatment interval of the non-dapansutrile treatment of the patient may be reduced.

The present invention is useful in treating a mammal subject, such as humans, horses, dogs and cats. The present invention is particularly useful in treating humans.

This application has demonstrated that dapansutrile treatment reduces tumor growth of E0771 TNBC in Tet2^{+/-} mice compared to those without dapansutrile treatment.

Moreover, the inventors have demonstrated bone marrow cells cultured from tumor-bearing Tet2^{+/-} mice exhibited increased IL-1β production compared to wild type mice. Furthermore, bone marrow cells from tumor-bearing Tet2^{+/-} mice treated with dapansutrile secreted significantly less IL-1β compared to untreated Tet2^{+/-} mice.

In triple-negative breast cancer, IL-1β promotes an immunosuppressive TME. The inventors have demonstrated that tumor-bearing Tet2^{+/-} mice producing more IL-1β in bone marrow adherent cells, which indicates that increases in Tet2+/− mutations lends the host more susceptible to an immunosuppressive TME. CHIP Patients with increases in Tet2 VAF are at a greater risk of advancing to late-stage disease, and dapansutrile intervention therapy inhibits the production of IL-1β and improves the patient condition. Dapansutrile is effective at reversing the CHIP phenotype by inhibiting the production of IL-1β and inhibits NLRP3 inflammasome activity.

This application has shown the tumor promoting outcomes of loss-of-function TET2 mutations in metastatic breast. Tet2-CH does not represent a specific disease, per se, but rather serves as a pre-loaded trigger worsening IL-1β mediated disease. The inventors have demonstrated a 2-fold increase in tumor growth in Tet2+/− germline mice. To determine if this observation was merely an artifact of Tet2 heterozygosity in the entire organism or truly a phenomena of Tet2 CH, the inventors generated BM chimeric mice using mixed BM with 25% Tet2 CH and showed that tumor growth increased 2-fold in Tet2+/− bone marrow chimeras compared to Tet2+/+ controls, which demonstrated that this was indeed a CH outcome.

Further, this application shows an NLRP3-dependent recruitment of myeloid cells to the tumor microenvironment (TME) through induction of plasma CCL2 and KC in Tet2+/− chimeras, resulting in increased tumor-infiltrating granulocytes. Consistently, the inventors observed a significant increase of donor-derived Tet2+/− mutants in the TME, thereby placing these hyperinflammatory cells in TME. These data demonstrate that recruitment of hyperinflammatory Tet2+/− myeloid cells to the breast cancer TME fuels breast cancer progression.

The addition of dapansutrile in the diet of Tet2+/− tumor bearing mice normalized the granulocyte levels to those found in Tet2+/+ mice, reflecting the dependence on IL-1β. These data implicate granulocytes in tumor progression and highlight the cycle of IL-1β induced granulopoiesis driving tumor growth. Therefore, dapansutrile presents a therapeutic approach to neutralizing inflammatory diseases mediated by Tet2+/−CH or DNMT3A CH.

The following examples further illustrate the present invention. These examples are intended merely to be illustrative of the present invention and are not to be construed as being limiting.

### EXAMPLES

The following materials and protocols were used in the examples described below.

### Mice

6-8 week old B6(Cg)-Tet2tm1.2Rao/J (JAX) female mice heterozygous for Tet2 allele or wild-type for Tet2 allele were purchased for Jackson Laboratories.

### Cell Lines

The murine metastatic mammary cancer cell line E0771 was purchased from ATCC. E0771 cells were cultured in RPMI (Corning) supplemented with 10% FBS, 1% HEPES, 100 units/ml penicillin and 0.1 mg/ml streptomycin. Cells were maintained in a humidified 5% CO₂ atmosphere at 37° C.

### Tumor Model

2×10⁵ E0771 cells were mixed with matrigel and implanted orthotopically into the mammary fat pad (day 0). Mice treated with dapansutrile were fed ad libitum with food pellets containing 7.5 g dapansutrile/kg food, which started on day 3 after E0771 cell implantation and continued until being sacrificed. Mice were sacrificed on day 18 for the germline mouse model and day 15 for the chimeric mouse model. Mice typically consume about 4 g of food per day, resulting in an approximate daily dose of 0 mg/kg/day for control groups and 1,000 mg/kg/day for the treatment groups. This food pellet concentration (7.5 g/kg of dapansutrile in food) in mouse chow resulted in a blood level nearly the same as that of humans treated orally with dapansutrile at doses of 1000 mg/day (40 μg/mL blood level) (14). Wild-type and Tet2+/− mice were fed with control food pellets without dapansutrile. Mice were sacrificed following 15-18 days after E0771 cell implantation. Tumor volumes were assessed using ½(L×W×H) measured via digital caliper.

Mice with Chimeric Bone Marrow (BM)

To create a mouse model that more closely resembles human CHIP, we generated mice with chimeric bone marrow (BM) that was 75% wild type (WT), and 25% Tet2 homozygous (Tet2+/+) or heterozygous (Tet2+/−). To identify the contribution of WT or Tet2+/− cells to hematopoiesis, we used the pan hematopoietic marker CD45. We used two congenic mouse strains with variants in CD45 C57BL/6 and Boy/J (CD45.2 and CD45.1, respectively), which can be identified with fluorescently labeled antibodies using flow cytometry analysis. To generate the chimeric BM, we lethally irradiated Boy/J recipient mice (CD45.1) with an 11gy split-dose administered three hours apart. Irradiated recipient mice were then transplanted with 2 million unfractionated BM cells that were a mix of 75% CD45.1 WT and 25% CD45.2

Tet2+/+ or Tet2+/−. Cells were given via a retro-orbital (RO) sinus injection. To monitor the engraftment of donor-derived cells, mice were bled every four weeks via the submandibular vein. Blood was then processed via hemolysis to remove red blood cells, and subsequently, the white blood cells were stained with fluorescent antibodies for flow cytometry analysis.

After 6 weeks of engraftment, mice were given breast tumors via orthotopic transplantation of breast cancer cells. Tumor progression was assessed by measuring the volume of the tumor in the mouse using calipers. After 15 days, mice were sacrificed, and their bone marrow, peripheral blood, and tumors were harvested for flow cytometry analysis to determine the levels of immune cell populations and relative contribution from WT or Tet2+/+ or Tet2+/− bone marrow cells.

### Bone Marrow Cytokine Production

Bone marrow cells from tumor-bearing mice was collected and filtered through 40 μM cell strainer. 3×10⁶ cells were plated in 24-well plates and incubated overnight. The next day to non-adherent fraction was removed and the adherent fraction was treated with 10 μM dapansutrile and were cultured in RPMI supplemented with 10% FBS, 100 units/ml penicillin and 0.1 mg/ml streptomycin for 24 hours. Cytokines were measured in cell culture supernatants using DuoSet ELISA (R&D Systems).

### Flow Cytometry

Bone marrow isolation and cell staining was performed on ice in staining medium (SM; Hanks Balanced Salt Solution [HBSS 1×, Corning 1-022-CM] supplemented with heat inactivated fetal bovine serum [FBS, VWR 97068-05] to a final concentration of 2%). Femurs and tibiae were flushed with 3 mL of SM and followed by RBC lysis with ACK lysis buffer. Peripheral blood was isolated via the heart and placed into RBC lysis buffer. After RBC lysis cells were incubated on ice for 30 minutes with antibody cocktails prior to being washed and resuspended in SM containing propidium iodide to stain dead cells. Flow cytometry was performed on the BD FACSCelesta.

### Adherent Bone Marrow Isolation, Culture and Stimulation

Bone marrow cells were collected from the four long bones as described above and filtered through a 40 μM cell strainer prior to counting. 1×10⁶ cells were plated in flat bottom tissue culture plates and incubated overnight. The next day the non-adherent fraction was removed and the adherent fraction was used for downstream experiments.

### Cytokine Analysis

For western blot and cytokine analysis, adherent bone marrow cells were stimulated±LPS (100 ng/mL) and cultured for an additional 24 hours. Cells were cultured in RPMI supplemented with 10% FBS, 100 units/ml penicillin and 0.1 mg/ml streptomycin. For conditioned media stimulation, E0771-conditioned media was collected by incubating 1×10⁶ cells for 72-hours, supernatant was collected and centrifuged to removed cellular debris. Conditioned media was then added at 1:2 to normal media for 24 hours. Cytokines were measured in cell culture supernatants using DuoSet ELISA (R&D Systems).

### Example 1A: Dapansutrile Reduced Breast Tumor Volume in Tet2+/− Mice (Germline)

Myeloid cells are the predominant source of tumor-promoting IL-1β in the breast cancer TME, and TET2 mutations have been reported in tumor-infiltrating leukocytes. Since myeloid cells from Tet2-deficient mice have increased IL-1β gene expression following inflammatory stimulus, we assessed whether mice heterozygous for Tet2 would exhibit more aggressive breast cancer phenotype. We orthotopically implanted the murine metastatic luminal B breast cancer cell line, E0771, into Tet2+/+ (WT) or Tet2+/− germline mice. Three days after implantation, mice were placed on a control chow diet or a nutritionally identical diet enriched with OLT1177® (dapansutrile) for 15 days. Mice were sacrificed following 21 days after E0771 cell implantation.

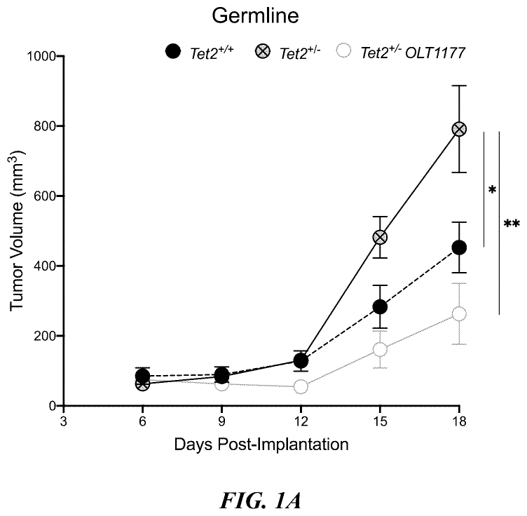

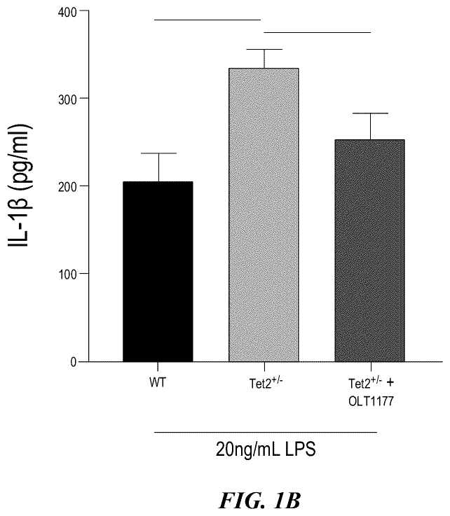

The results of tumor volume were shown in FIG. 1. Tet2^{+/-} mice exhibited a 2-fold increase in tumor growth compared to Tet2+/+ (WT) mice (p<0.05). Further, tumor growth was significantly suppressed in Tet2+/− mice receiving dapansutrile diet compared to Tet2^{+/-} mice on standard diet (68% reduction, p<0.01). Tumor growth in Tet2+/+ (WT) mice on dapansutrile was also significantly decreased compared to standard diet (p<0.05). No changes in tumor growth were observed between Tet2+/+ (WT) or Tet2+/− mice on dapansutrile diet (FIG. 1).

### Example 1B: Dapansutrile Reduced IL-1β Production in Tet2+/− Mice

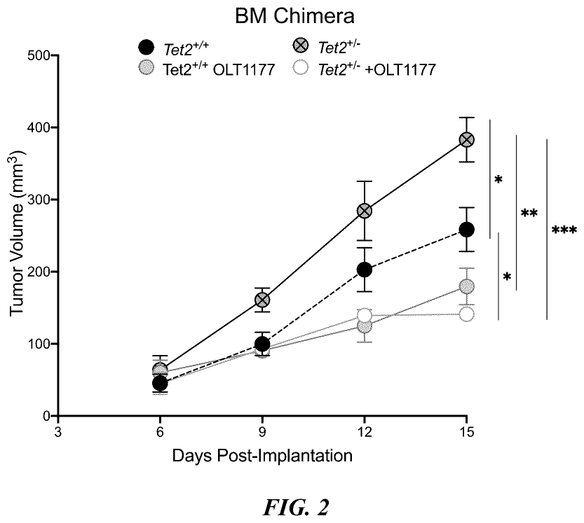

Next, we assessed the inflammatory potential of myeloid cells from tumor-bearing mice. Bone marrow adherent cells were cultured from tumor-bearing mice of Example 1 and stimulated with LPS overnight. Bone marrow cells cultured from tumor-bearing Tet2+/− mice exhibited increased IL-1β production compared to WT (p<0.01, FIG. 1B). Furthermore, adherent bone marrow cells from tumor-bearing Tet2+/− mice treated with dapansutrile secreted significantly less IL-1β compared to untreated Tet2+/− mice (p<0.05, FIG. 2).

### Example 2: Dapansutrile Reduced Breast Tumor Volume in a Tet2+/− Bone Marrow Chimeric Model

CH is characterized by clonal expansion of somatic mutations in the hematopoietic compartment. In this example, we determined whether the tumor-promoting role of Tet2 deficiency was maintained in a bone marrow chimeric model (experimental model for clonal hematopoiesis).

We mixed unfractionated bone marrow (BM) cells from Tet2+/+ and Tet2+/− donor mice with CD45.1+ Boy/J competitor BM at a 1:3 donor: competitor ratio and transplanted the cells into lethally irradiated Boy/J recipient mice. Six weeks after engraftment, E0771 cells were orthotopically implanted as described above. On day 3 after implantation, mice were given either a control chow or dapansutrile enriched diet. We observed an essentially identical pattern of accelerated tumor growth in Tet2+/− chimeric mice (p<0.05), which was again suppressed in Tet2+/− chimeric mice receiving dapansutrile diet (p<0.001). Likewise, dapansutrile also suppressed tumor growth in Tet2+/+ chimeric animals, to a baseline level equivalent to dapansutrile-treated Tet2+/− chimeric mice (FIG. 2). The results of Examples 1 and 2 indicate that Tet2+/− promotes breast cancer progression, and dapansutrile is effective to reduce the tumor growth.

### Example 3. Accelerated TET2+/− Driven Breast Tumor Growth is Reduced by Dapansutrile

Granulocytes and monocytes, collectively called myeloid cells, are differentiated descendants from common progenitors derived from hematopoietic stem cells in the bone marrow.

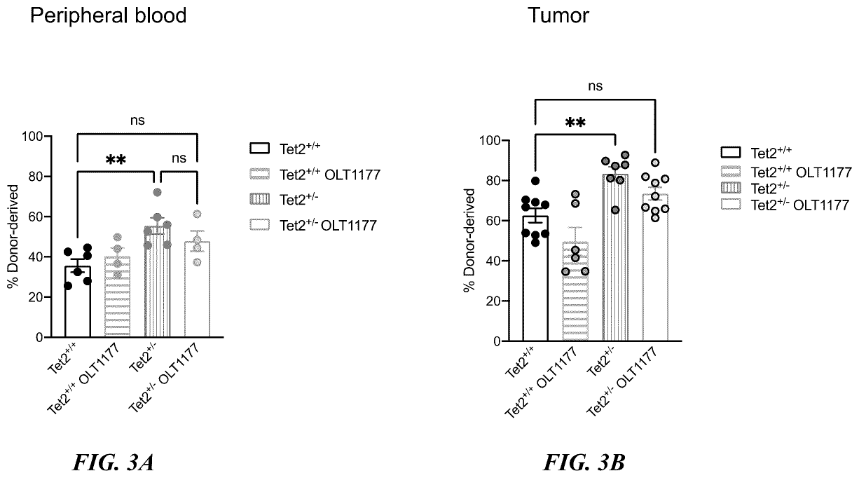

In this example, we first assessed the extent to which Tet2-deficient myeloid cells infiltrated into peripheral blood (FIG. 3A) and in the tumor (FIG. 3B). FIG. 3A shows that the frequency of donor-derived cells was significantly increased in the peripheral blood of Tet2+/− chimeric mice, comparing with Tet2+/+ mice. FIG. 3B shows that the frequency of Tet2+/− donor-derived cells was significantly increased in tumors of Tet2+/− chimeric mice, comparing with Tet2+/+ mice.

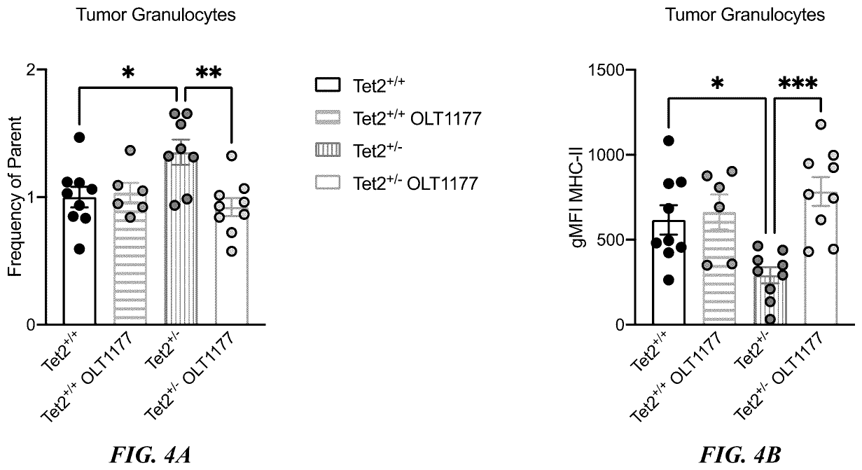

FIG. 4A shows that total tumor-infiltrating granulocytes significantly increased in Tet2+/− chimeras receiving standard diet compared to Tet2+/+ chimeras receiving standard diet (p<0.05). FIG. 4A also shows that tumor-infiltrating granulocytes were significantly decreased in Tet2+/− chimeras receiving dapansutrile diet compared to Tet2+/− chimeras on standard diet (p<0.05).

FIG. 4B shows tumor-infiltrating granulocytes expressed significantly less MHC-II protein (gMFI) in Tet2+/− chimeras receiving standard diet compared to Tet2+/+ chimeras receiving standard diet (p<0.05). The reduced MHC-II expression found in Tet2+/− chimeras was rescued with dapansutrile diet (p<0.001).

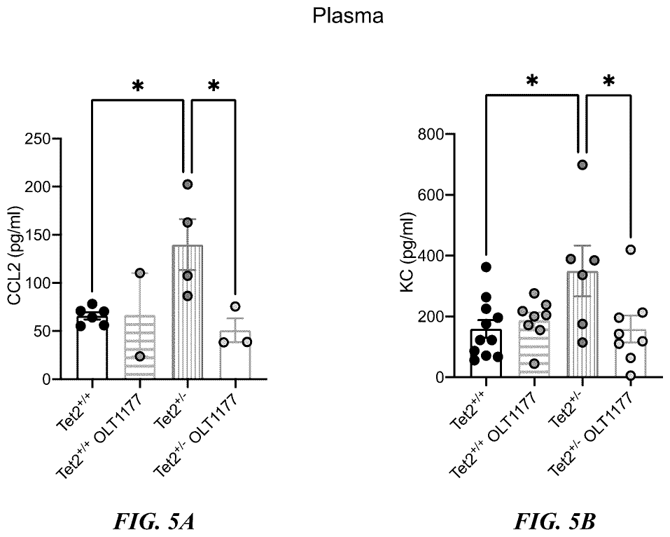

FIG. 5 shows plasma chemokines and cytokines in BM chimera mice from FIG. 2. As shown in FIG. 5, Tet2+/− chimeric mice receiving standard diet revealed increased circulating CCL2 and KC compared to Tet2+/+ chimeras on standard diet (p<0.05). Further, circulating CCL2 and KC were significantly decreased in Tet2+/− chimeras receiving DAPANSUTRILE diet compared to Tet2+/− chimeras on standard diet (p<0.05).

Taken together, these data demonstrate how breast cancer-promoted expansion of Tet2+/− clones perpetuates the inflammatory environment of the TME, in turn fueling breast cancer progression, and dapansutrile was able to reduce the expansion of Tet2+/− clones.

### REFERENCES

1. Y. Guo, et al. Cancer Res 77, 6429-6441 (2017).
2. S. Shalapour, et al. J Clin Invest 125, 3347-3355 (2015).
3. C. A. Dinarello. Blood 117, 3720-3732 (2011).
4. R. N. Apte, et al. Cancer Metastasis Rev 25, 387-408 (2006).
5. C. A. Dinarello. Cancer Metastasis Rev 29, 317-329 (2010).
6. B. Guo, et al. Sci Rep 6, 36107 (2016).
7. K. L. Lee, et al. Cancers 11 (9), 1334 (2019).
8. C. Marchetti, et al. Proc Natl Acad Sci USA 115, E1530-E1539 (2018).
9. A. Young, et al. Nature Communications. DOI: 10.1038 (2016)
10. L. Dorsheimer, et al. JAMA Cardiol. 4:25-33 (2019)
11. N. L. Syn, et al. Lancet Oncology 18 (12), PE731-E741 (2017).

From the foregoing, it will be appreciated that specific embodiments of the invention have been described herein for purposes of illustration, but that various modifications may be made without deviating from the scope of the invention. Accordingly, the invention is not limited by the appended claims.

What is claimed is:

1. A method of treating a solid cancer in a patient, comprising the steps of:
(a) determining clonal mutation of TET2 or DNMT3A in a sample of a patient that has a solid cancer of breast cancer or prostate cancer and is treated with a non-dapansutrile treatment, and
(b1) treating the patient with an effective amount of dapansutrile in addition to the non-dapansutrile treatment, if the patient is determined to have a variant allele frequency (VAF) of TET2 or DNMT3A greater than 0.02, or (b2) continuing treating the patient with the non-dapansutrile treatment, if the patient is determined to have a VAF of TET2 or DNMT3A≤0.02.

2. The method of claim 1, wherein the solid cancer if breast cancer, and the non-dapansutrile treatment is check point inhibitor treatment.

3. The method of claim 2, wherein said check point inhibitor is an inhibitor to programmed cell death protein 1 (PD-1), programmed death ligand 1 (PD-L1), and cytotoxic T lymphocyte associated protein 4 (CTLA-4).

4. The method of claim 3, wherein said check point inhibitor is an inhibitor to PD-1.

5. The method of claim 4, wherein said check point inhibitor is an anti-PD-1 antibody.

6. The method of claim 2, wherein the non-dapansutrile treatment is a chemotherapy.

7. The method according to claim 2, wherein the breast cancer is selected from the group consisting of: ductal carcinoma in situ (DCIS), invasive ductal carcinoma (IDC), triple negative breast cancer (TNBC), inflammatory breast cancer (IBC), metastatic breast cancer, and breast cancer during pregnancy.

8. The method of claim 1, further comprising a step of selecting a patient that has breast cancer and is treated with a non-dapansutrile treatment before step (a).

9. The method according to claim 1, wherein dapansutrile is administered by oral administration.
